(19) United States
(12) Patent Application Publication
(10) Pub. No.: US 2007/0059345 A1
(43) Pub. Date: Mar. 15, 2007

(54) Method for tissue growth

(76) Inventor: Craig McLachlan, Westleigh (AU)

Correspondence Address: OLSON & HIERL, LTD., 20 NORTH WACKER DRIVE, 36TH FLOOR, CHICAGO, IL, 60606, US

(21) Appl. No.: 10/554,348

(22) PCT Filed: Apr. 23, 2004
(86) PCT No.: PCT/AU04/00538

§ 371 Date: Aug. 17, 2006

(30) Foreign Application Priority Data
Apr. 24, 2003 (AU) 2003902037

(51) Int. Cl. C12N 5/08; A61K 9/70

(52) U.S. Cl. 424/443; 435/366

(57) ABSTRACT

A method for promoting or stimulating growth of tissue (22) comprising providing an element (23) which is adapted to receive blood, locating the element (23) in contact with the tissue (23) with at least one portion of the element (23) extending away from an external surface of the tissue (22), and conditioning the element (23) by introducing blood into said at least one portion whereby, when located in contact with the tissue, the conditioned element (23) is arranged to stimulate or promote growth of tissue in (27) and from (28) the at least one portion.

### FIELD OF THE INVENTION

The invention relates to the use of an element in tissue growth for treatment of conditions, particularly, for treatment of diseases associated with reduced vascularisation.

### BACKGROUND OF THE INVENTION

The growth and maintenance of healthy tissue is dependent on vascularisation within the tissue to provide the necessary requirements for constituent cell growth and maintenance of the tissue. Consequently, circumstances which lead to depletion or loss of vascularisation may lead to reduction in blood flow to the tissue and reduced tissue function.

Diseases that result from a reduction of blood flow and as a result, reduced tissue function, constitute a significant health problem in industrialised countries. For example, ischaemic heart disease results from depleted blood flow in the heart muscle. The loss of blood flow to regions of the heart muscle may result in reduction of heart muscle function, or damage to the heart muscle as is the case in diseases such as angina (stable or unstable), pre-infarction angina, myocardial infarction, heart failure or in patients with cardiac pacing.

There is a need for a method for tissue growth to provide improved vascularisation for tissue and thereby provide an improved treatment for diseases associated with reduced vascularisation such as ischaemic heart disease, myocardial infarction and artheriosclerosis.

### SUMMARY OF THE INVENTION

In one aspect, the invention provides a method for promoting or stimulating growth of tissue comprising the following steps:
providing an element which is adapted to receive blood;
locating the element in contact with the tissue with at least one portion of the element extending away from an external surface of the tissue;
conditioning the element by introducing blood into said at least one portion, whereby when located in contact with the tissue, the conditioned element is arranged to stimulate or promote growth of tissue in and from the at least one portion.

As described herein, the inventor has found that conditioning an element by introducing blood into at least one portion of the element that is in contact with a tissue can stimulate or promote tissue growth in and from the at least one portion. Such tissue growth includes the stimulation or promotion of growth of blood vessels in and from the at least one portion. The stimulation or promotion of growth of blood vessels may include the stimulation or promotion of angiogenesis and/or arteriogenesis in and from the at least one portion. As used herein, “angiogenesis” refers to the growth of blood vessels having a diameter of less than 200 micrometres. As used herein, “arteriogenesis” refers to growth of blood vessels having a diameter of at least 200 micrometres. The blood vessels formed by arteriogenesis suitably comprise smooth muscle cells. The method therefore provides a means for re-vascularisation. In particular, the method provides a means for the treatment of tissue in need of re-vascularisation such as, for example, treatment of ischeamic tissue, treatment of blockages in blood vessels, or to increase vascularisation in normal healthy tissue or healthy tissue at risk of ischaemia. The method also provides a means for the growth of tissue such as pancreatic tissue. Additionally, the method provides a means to reduce tissue loss, for example, from the cross-sectional wall of the aorta when the element is wrapped around the aorta.

The method has the advantage that tissue can be grown in and from locations previously not thought possible because these locations lack support structure and blood supply. The inventor has found that by introducing blood into the at least one portion, tissue growth is stimulated or promoted from the at least one portion, thereby making it possible using the method of the invention to grow tissue in locations depleted of tissue or blood supply.

Further, the inventor has found that the tissue that grows from the at least one portion of the element may include large blood vessels, thereby making it possible using the method of the invention to improve collateral circulation in tissue in need thereof.

The element may be located at any location where tissue growth, preferably vascularisation, is required. Suitably at least one surface of the element is in contact with the external surface of the tissue. For example, by locating the element with at least one surface of the element in contact with the external surface of the tissue, growth of tissue may be promoted or stimulated from the at least one portion onto the surface of the tissue. Growth of the tissue may further extend into the tissue. In other words, by locating at least one surface of the element over and in contact with a portion of the tissue external surface, growth of tissue, and in particular angiogenesis and/or arteriogenesis, can be promoted or stimulated in and from the at least one portion into the tissue external surface in contact with the at least one portion, leading to increased vascularisation of the tissue in contact with the at least one portion. In addition, tissue may grow from the element that does not extend into the tissue external surface.

The tissue which grows in and from the element may comprise large blood vessels. As used herein, “large blood vessels” have a maximum diameter that is greater than that of the maximum diameter of capillary blood vessels. The maximum diameter of the large blood vessels may be at least 150 micrometres in diameter. The maximum diameter of the large blood vessels may be at least 200 micrometres in diameter. The large blood vessels may have well-developed adventitia. The large blood vessels may result from angiogenesis and/or arteriogenesis.

The conditioned element may stimulate or promote:
angiogenesis in and from the element;
arteriogenesis in and from the element;
angiogenesis and arteriogenesis in and from the element;
angiogenesis in the element and arteriogenesis from the element; and/or
arteriogenesis in the element and angiogenesis from the element.

In one embodiment, at least one surface of the element is in contact with the external surface of ischaemic tissue. The ischaemic tissue may be ischaemic cardiac tissue. The cardiac tissue may be, for example, infarcted tissue, fibrotic tissue or scar tissue. However, it will be appreciated by persons skilled in the art that the type of tissue may be any tissue, including for example, pancreatic tissue, aortic tissue, liver tissue, bladder tissue, bone tissue or neural/nerve tissue. It will also be appreciated by persons skilled in the art that the tissue may be tissue that has normal blood and nutrient perfusion. The tissue may also be tissue that is non-ischaemic tissue, but which is of risk of ischaemia, or in a person at risk of ischaemia.

The blood may be introduced into the at least one portion by any means. In one embodiment, the blood is introduced by drawing the blood into the at least one portion. Preferably, the blood is drawn into the at least one portion by the element. The blood may be drawn, for example, by absorption by the element or by capillary action of the element. The blood may alternatively be introduced by applying suction to the element, or by pumping or working the blood into the element, or by soaking or dowsing the element in blood from a wound or vascular blood service.

The blood may be introduced into the at least one portion from one or more sites. The blood may be introduced in to the at least one portion from at least one wound. In one embodiment, the at least one wound is formed in the tissue. In one embodiment, the at least one wound is a channel formed in the tissue.

In one embodiment, the wound is formed in the tissue by tearing or teasing apart the tissue. In another embodiment, the wound may be formed in the tissue by cutting. The cut may be formed using any conventional surgical cutting technique, for example, incision, drilling or boring, abrasion etc. Typically the tissue is cut by incising the tissue. The incision may be formed using any means capable of forming the incision, including for example, a scalpel or surgical knife or the like, or a laser. For example, a laser for use in trans myocardial laser revascularisation (TMLR) or a mechanical channelling device may be used to form the wound.

The at least one portion may be in contact with the at least one wound, or remote but in fluid communication with the at least one wound. In one embodiment, the at least one portion is in contact with the at least one wound. For example, the at least one portion may be placed over the at least one wound to receive blood from the wound to thereby promote or stimulate tissue growth in and from the at least one portion. The at least one portion may be flooded or bathed in blood from the wound. A device that is capable of promoting or stimulating angiogenesis within the wound may be inserted in the wound whereby tissue growth is promoted or stimulated between the at least one portion and the device. The device may be in contact with the at least one portion, or may be spaced apart from the at least one portion.

The at least one wound may be in contact with one or more other portions of the element that are remote to, and in fluid communication with, the at least one portion. Thus, in another embodiment, the at least one portion may be in fluid communication with the one or more other portions of the element, which in turn are in contact with the at least one wound so that blood is introduced into the at least one portion via the one or more other portions. In one embodiment, the at least one portion is in fluid communication with the one or more portions via interconnecting pores of the element.

The element may be of any shape. For example, the element may be sheet like or elongate in shape. The element may comprise a recess for insertion of cells or compounds into the element or for direct passage of fluid. The element may be recessed along a substantial portion of the length of the element to create a channel for passage of blood into the element.

In one embodiment, the at least one portion of the element is a sheet-like structure having at least one surface of the at least one portion in contact with a portion of the external surface of the tissue.

In another embodiment, the element is an elongate member having at least one surface of the least one portion in contact with a portion of the external surface of the tissue.

In yet another embodiment, the element has at least one surface of the at least portion of the element in contact with the wound.

In yet another embodiment, the element has at least one surface of the at least one portion in contact with a portion of the external surface of the tissue, and at least one other portion in contact with the wound to receive blood from the wound, the at least one other portion being in fluid communication with the at least one portion.

The at least one surface of the at least one portion may contact the external surface of the tissue between at least two wounds whereby growth of tissue from the at least one portion is stimulated or promoted in and from the at least one portion. For example, growth of blood vessels may be stimulated or promoted between two wounds wherein one of the wounds is close to an occluded artery to stimulate growth of blood vessels to the occluded artery, and/or to tissue nearby the occluded artery, to thereby restore blood flow.

Typically, the element comprises a porous structure with a pore size sufficient for permitting tissue growth in and through pores of the porous structure. In particular, the pore size will be optimal for blood vessel growth in and through the element. Typically, the average size of the pores of the element are less than 250 microns in diameter. The average size of the pores may range from 50 to 250 microns in diameter. The porous structure may comprise interconnecting pores. The element may be sponge-like. The element may be capable of fluid absorption.

The void content of the porous structure, or in other words, the proportion of the volume of the element that is pore space, is typically between 50% and 90% of the total volume of the element. This void content is optimal for blood vessel growth in and through the element. In one embodiment, the void content is between 70% and 90% of the total volume of the element.

Typically, the element has the compliance of a polyurethrane. The element may be formed from a polyurethane, a polyether urethane, a polyether urethane urea, a polyether carbonate urethane, a polyether carbonate urethane urea, a polycarbonate urethane, a polycarbonate urethane urea, polycarbonate silicone urethane, a polycarbonate silicone urethane urea, a polydimethylsiloxane urethane, a polydimethylsiloxane urethane urea, a polyester urethane, a polyester urethane urea, pellethane, chronoflex, hydrothane, estane, Elast-Econ, Texin, Biomer type polyurethanes, Surethane, Corethane, Carbothane, Carbonate, Techoflex, Techothane and Biospan, or mixtures thereof. The element may also have the compliance of a polyurethane as described in Ziller Peter Paul et al. U.S. Ser. No. 20010002444, U.S. Pat. No. 6,245,090 or U.S. Pat. No. 6,177,522.

Typically, the element comprises at least one polyurethane described above.

The element may comprise absorbable or non-absorbable suture materials, fibre or yarn such as those that are typically used in surgical and/or wound and/or tissue engineering applications (see, for example, U.S. Pat. Nos. 6,638,284; 3,054,406; 3,124,136; 4,193,137; 4,347,847). These materials may be those formed in woven mats or meshes. These suture materials, fibres or yarns may be braided, knitted or weaved to define the element. The absorbable or non-absorbable suture materials may be comprised with the above described polyurethanes.

In one embodiment, the element comprises one or more absorbable compounds. Examples of absorbable compounds include elastin, tropoelastin, collagen, starch, fibrin, polyhydroxyalkanoate, poly(1,3-trimethylene carbonate), tofu, caprolactone-c-L-lactide, knitted poly-L-lactide fabric or poly(glycerol-sebacate) or mixtures thereof. Non-absorbable suture material, fibre or yarn may be formed, for example, from one or more of the following materials: cotton, linen, silk, knitted silkworm silk, insect silk, wool, rayon, acetate, aramids (eg. Kevlar, Numex), polyphenylene sulfide, polyester, polyamides (polyhexamethylene adipamide (nylon 66), polyhexamethylene sebacamide (nylon 610), polycapramide (nylon 6), polydodecanamide (nylon 12) and polyhexamethylene isophthalamide (nylon 61) copolymers and blends thereof), polyesters (e.g. polyethylene terephthalate, polybutyl terephthalate, copolymers and blends thereof), fluoropolymers (e.g. polytetrafluoroethylene and polyvinylidene fluoride) polyolefins (e.g. polypropylene, including isotactic and syndiotactic polypropylene and blends thereof, as well as, blends composed predominately of isotactic or syndiotactic polypropylene blended with heterotactic polypropylene (such as are described in U.S. Pat. No. 4,557,264 issued Dec. 10, 1985 assigned to Ethicon, Inc. hereby incorporated by reference) and polyethylene.

Absorbable suture material, fibre or yarn may be formed, for example, from one or more of the following materials: aliphatic polyesters which include but are not limited to homopolymers and copolymers of lactide (which includes lactic acid d-, l- and meso lactide), glycolide (including glycolic acid), epsilon-caprolactone, p-dioxanone (1,4-dioxan-2-one), trimethylene carbonate (1,3-dioxan-2-one), alkyl derivatives of trimethylene carbonate, delta-valerolactone, beta-butyrolactone, gamma-butyrolactone, epsilon-decalactone, hydroxybutyrate, hydroxyvalerate, 1,4-dioxepan-2-one (including its dimer 1,5,8,12-tetraoxacyclotetradecane-7,14-dione), 1,5-dioxepan-2-one, 6,6-dimethyl-1,4-dioxan-2-one and polymer blends thereof, polyglactin 910 (Vicryl), polyglycolic acid (Dexon).

In one embodiment, the element comprises a mesh formed from knitted or woven suture material, yarn or fibre, wherein the mesh is coated with polyurethane, polyurethane polycarbonate or polyether urethane.

The suture material, fibre or yarn may be coated in polyurethane. For example, by immersing the suture material, yarn or fibre in, or spraying with, a solution of one or more of any of the abovementioned polyurethanes, the suture material or fibres or yarn may be suitably coated with the polyurethanes to provide an element that can be used in the method of the invention. The suture material or fibres or yarn may be coated with polyurethane prior to, during, or subsequent to, weaving or knitting of the suture material or fibres or yarn into a mat or mesh.

For example, the woven or knitted mats or meshes may be treated or coated with a polyurethane polycarbonate or a polyether urethane. The coating can be applied by dipping the mat or mesh into a solution of the polyurethane polycarbonate, or by spraying the mat or mesh with the solution of polyurethane polycarbonate, and allowing it to dry according to the manufactures instructions (methods for coating with polyurethane polycarbonate are provided in, for example, Kim D H, Kang S G, Choi J R, Byun J N, Kim Y C, Ahn Y M. Evaluation of the biodurability of polyurethane-covered stent using a flow phantom. Korean J Radiol. 2001 April-June; 2(2):75-9). Suitable commercially available polyurethane polycarbonates include Carbothane PC3570A, Chronoflex, Corethane 80A, and Corethane 55D. Suitably, the polyurethane polycarbonate is Chronoflex (which is available commercially from CardioTech International, Inc.).

The tissue for treatment may be any tissue. The tissue may be muscle tissue, such as cardiac muscle tissue. The tissue may be, for example, pancreatic tissue, or blood vessel tissue such as an aorta.

The element may further comprise at least one agent for controlling growth of tissue in and from the at least one portion. In one embodiment, the at least one portion comprises at least one agent for controlling growth of tissue in and from the at least one portion. The agent may be one capable of controlling regeneration of the tissue, or capable of controlling fibrosis, or formation of scar tissue. The agent may promote or stimulate regeneration of the tissue. Examples of such agents include: epidermal growth factor agonists, transforming growth factor-beta antagonists (1,2 and 3), IGF, TGF-α, VEGF, FGF, β-FGF, GAS-6, PDGF, IGF binding protein, platelet-derived growth factor antagonists, angiotensin converting enzyme (ACE), Ang II receptor antagonists [such as AT1 (losartan) or AT2 (PD123177)], inhibitors of plasminogen activators, inhibitors of matrix metalloproteinases, inhibitors of collagen prolyl hydroxylase, inhibitors of urokinase-type plasminogen activator, Bradykinin B2 receptor antagonists (for example, Hoe 140), inhibitors of cyclooxygenase (for example, indomethacin), calmodulin antagonists, anesthetics such as lidocaine and pentobarbital, inhibitors of polymorphonuclear leukocyte elastase and inhibitors of leukocyte migration, or mixtures thereof.

The element may further comprise at least one species of cell for growth of tissue in and from the at least one portion. In one embodiment, the at least one portion further comprises at least one species of cell for growth of tissue in and from the at least one portion. Examples of such cells include endothelial cells, smooth muscle cells, skeletal muscle cells, pericytes, embryonic stem cells, stem cells, bone marrow, cultured myocytes or precursors of cardiomyocytes, myofibroblasts, fibroblasts and cells expressing proteins to promote angiogenesis, and/or arteriogenesis, and/or cell growth.

The element, or the at least one portion, may comprise cells from a source other than the tissue on which the element is to be arranged. The element, or the at least one portion, may be impregnated with cells prior to the arrangement of the element on the tissue. Alternatively, the element, or the at least one portion, may be impregnated with the cells subsequent to arrangement on the tissue.

The element may comprise at least one agent for attracting cell types to the element. Suitably, the agent for attracting cells to the element is capable of attracting cells such as stem cells, resident satellite cells. Suitable agents for attracting cell types to the element include chemotaxins or receptors. An example of a chemotaxin suitable for attracting stem cells to the element is stromal cell-derived factor-1 (SDF-1). An example of a receptor that is suitable for attracting stem cells to the element is the stromal cell-derived factor-1 receptor (CXCR-4).

The element may further comprise at least one agent for controlling angiogenesis and/or arteriogenesis in and from the at least one portion. In one embodiment, the at least one portion further comprises at least one agent for controlling angiogenesis and/or arteriogenesis in and from the at least one portion. Typically the agent promotes or stimulates angiogenesis throu ghout the element. Examples of such agents include: erythropoietin (Epo), recombinant human Epo, IGF, TGF-α, TGF-β, VEGF, FGF, β-FGF, GAS-6, PDGF, PIGF, IGF binding protein, EGF, TNF, IL-8, IL-β, heparin, warfarin, inhibitors of matrix metalloproteinases, agonists of matrix metalloproteinases, simvastatin, nicotinic analogues, nicotinic agonists, nicotinic antagonists, angiopoiten, dopamine analogues, dopamine agonists, dopamine antagonists, other cytokines and serine proteases or mixtures thereof.

The element may further comprise bacteria or fragments thereof that are capable of promoting or stimulating angiogenesis. In one embodiment, the at least one portion further comprises bacteria or fragments thereof that are capable of promoting or stimulating angiogenesis. For example, the element or at least one portion may comprise bacteria, fragments of bacteria, heat killed bacteria, attenuated bacteria that are capable of promoting or stimulating angiogenesis, and/or wound healing, and/or tissue growth, and/or tissue repair. The bacteria themselves or their inter-action with macrophages, monocytes or endothelial cells may promote angiogenesis, prevent endothelial apoptosis and promote tissue proliferation and formation of blood vessels. Examples of such bacteria include Bartonella bacilliformis; B Henseale; Lactobacillus and H. pylori.

The element, or the at least one portion of the element, may also contain endothelial cells, monocytes, macrophages that interact with the bacteria, or fragments thereof, to promote angiogenesis and/or arteriogenesis.

The element, or the at least one portion of the element, may also contain macrophage chemo-attractant protein-1 (MCP-1) to recruit macrophages to interact with the bacteria.

The element, or the at least one portion of the element, may further comprise a combination of growth factors and or cytokines and or macrophage recruitment agents that would mimic and or enhance a response to the bacteria contained or impregnated to or within the element. An example of combined growth factors/cytokines would be VEGF, angiopoetin-2, TNF, IL-8, IL-1 beta. An example of a macrophage recruitment protein would be macrophage chemo-attractant protein-1 (MCP-1).

In another embodiment the channel contains extracellular matrix material and/or hydrogels that is viscous and permits oxygen and/or nutrient and/or blood to the element that it communicates with. The extracellular-matrix material within the channel may also contain growth factors and or stem cells and or stem cell homing factors to support as mentioned above angiogenesis and or collateral arteriogenesis and or tissue growth in and or around the channel. These added components to and within the channel may also benefit the sponge-like element with migration of stem cells to the element; growth factors and nutrient to promote tissue growth, angiogenesis and or arteriogenesis. Examples of suitable extracellular matrix material include

collagen, laminin, fibronectin, tropoelastin (such as, for example, recombinant tropoelastin), vitronectin, or combinations or mixtures thereof.

In another aspect, the invention provides a method for promoting or stimulating growth of tissue comprising the following steps:
providing an element which is adapted to receive blood;
locating the element in contact with the tissue with at least one portion of the element extending away from an external surface of the tissue;
conditioning the element by introducing blood into said at least one portion whereby when located in contact with the tissue, the conditioned element is arranged to stimulate or promote angiogenesis in and from the at least one portion.

In another aspect, the invention provides a method for promoting or stimulating growth of tissue comprising the following steps:
providing an element which is adapted to receive blood;
locating the element in contact with the tissue with at least one portion of the element extending away from an external surface of the tissue;
conditioning the element by introducing blood into said at least one portion whereby when located in contact with the tissue, the conditioned element is arranged to stimulate or promote arteriogenesis in and from the at least one portion.

In another aspect, the invention provides a method for promoting or stimulating growth of tissue comprising the following steps:
providing an element which is adapted to receive blood;
locating the element in contact with the tissue with at least one portion of the element extending away from the external surface of the tissue;
conditioning the element by introducing blood into said at least one portion whereby when located in contact with the tissue, the conditioned element is arranged to stimulate or promote angiogenesis and arteriogenesis in and from the at least one portion.

In another aspect, the invention provides a method for promoting or stimulating growth of tissue comprising the following steps:
providing an element which is adapted to receive blood;
locating the element in contact with the tissue with at least one portion of the element extending away from the external surface of the tissue;
conditioning the element by introducing blood into said at least one portion whereby when located in contact with the tissue, the conditioned element is arranged to stimulate or promote angiogenesis in, and arteriogenesis from, the at least one portion.

In another aspect, the invention provides a method for promoting or stimulating growth of tissue comprising the following steps:
providing an element which is adapted to receive blood;
locating the element in contact with the tissue with at least one portion of the element extending away from an external surface of the tissue;
conditioning the element by introducing blood into said at least one portion whereby when located in contact with the tissue, the conditioned element is arranged to stimulate or promote arteriogenesis in, and angiogenesis from, the at least one portion.

In another aspect, the invention provides a method for promoting or stimulating growth of tissue comprising the following steps:
providing an element which is adapted to receive blood;
locating the element in contact with the tissue with at least one portion of the element extending from the tissue surface;
conditioning the element by introducing blood into said at least one portion whereby when located in contact with the tissue, the conditioned element is arranged to stimulate or promote growth of tissue in and from the at least one portion.

In another aspect, the invention provides a method for treating ischaemic cardiac tissue. The method comprises the following steps:
providing an element which is adapted to receive blood;
locating the element with at least one portion of the element in contact with the epicardial external surface of the ischaemic cardiac tissue;
conditioning the element by introducing blood into
the at least one portion wherein the growth of tissue is stimulated or promoted in and from the at least one portion into the ischeamic cardiac tissue.

In one embodiment, the blood is introduced from a wound. The wound may be a channel extending through at least a portion of the myocardial wall. The channel may extend completely through the myocardial wall. The channel may be in ischaemic cardiac tissue or non-ischaemic cardiac tissue.

In another aspect, the invention provides a method for treating ischaemic heart disease. The method comprises
providing an element which is adapted to receive blood;
locating the element with at least one portion of the element in contact with the epicardial external surface of ischaemic cardiac tissue;
conditioning the element by introducing blood into
the at least one portion wherein the growth of tissue is stimulated or promoted in and from the at least one portion into the ischeamic cardiac tissue.

In one embodiment, the blood is introduced from a wound. Preferably, the wound may be a channel extending through at least a portion of the myocardial wall. The channel may extend completely through the myocardial wall. The channel may be in ischaemic cardiac tissue or non-ischaemic cardiac tissue.

In another aspect, the invention provides a method for treating myocardial infarction. The method comprises
providing an element which is adapted to receive blood;
locating the element with at least one portion of the element in contact with the epicardial external surface of the infarcted tissue;
conditioning the element by introducing blood into said at least one portion to stimulate or promote growth of tissue in and from the at least one portion into the infarcted tissue and/or infarct tissue border zone.

In another aspect, the invention provides a method for treating a thinning cross-sectional myocardial infarct scar. The method comprises
providing an element which is adapted to receive blood;
locating the element with at least one portion of the element in contact with the epicardial external surface of the infarcted scar tissue;
conditioning the element by introducing blood into said at least one portion to stimulate or promote growth of tissue in and from the at least one portion into the infarcted tissue and/or mature infarct scar.

In one embodiment, the blood is introduced from a wound. The wound may be a channel extending through at least a portion of the myocardial wall. The channel may extend completely through the myocardial wall. The channel may be in infarcted tissue or non-infarcted tissue.

In another embodiment, the method of the invention may be used to treat arrythmias due to abnormal electrical conduction in the heart muscle.

In another aspect, the invention provides a growth promoting or stimulating element when used in the method of the invention.

In another aspect, the invention provides a growth promoting or stimulating element adapted to receive blood such that, when the element is located in contact with tissue with at least one portion extending away from an external surface of the tissue, and conditioning the element by introducing blood into at least one portion of the element, growth of tissue is promoted or stimulated in and from the at least one portion of the element.

In another aspect, the invention provides a growth promoting or growth stimulating element comprising a mesh formed from knitted or woven yarn or fibre, wherein the mesh is coated with polyurethane or polyurethane polycarbonate.

The yarn or fibre of the growth promoting or stimulating element may be formed from one or more materials selected from the group consisting of cotton, linen, silk, knitted silkworm silk, insect silk, polyamides, polyhexamethylene sebacamide, polycapramide, polydodecanamide, polyhexamethylene isophthalamide, polyesters, fluoropolymers, polyolefins and polyethylene.

The yarn or fibre of the growth promoting or stimulating element may be formed from one or more materials selected from the group consisting of aliphatic polyesters, glycolide (including glycolic acid),. epsilon.-caprolactone, p-dioxanone (1,4-dioxan-2-one), trimethylene carbonate (1,3-dioxan-2-one), alkyl derivatives of trimethylene carbonate, delta.-valerolactone, beta.-butyrolactone, gamma.-butyrolactone, .epsilon.-decalactone, hydroxybutyrate, hydroxyvalerate, 1,4-dioxepan-2-one (including its dimer 1,5,8,12-tetraoxacyclotetradecane-7,14-dione), 1,5-dioxepan-2-one, 6,6-dimethyl-1,4-dioxan-2-one and polymer blends thereof. polyglactin 910 (Vicryl) and polyglycolic acid (Dexon)

The growth promoting or growth stimulating element may be coated with any of the polyurethane or polyurethane polycarbonates mentioned above.

The invention will be more fully understood from the following description of the various embodiments of the method of the invention and examples of support elements for use in performing the method of the invention.

### BRIEF DESCRIPTION OF THE DRAWINGS

FIG. 1 illustrates a schematic representation of cross-sectional area of a myocardial ventricular wall with an occluded and non-occluded coronary artery.

FIG. 2 illustrates an embodiment of the method of the invention in which a sponge-like element is located on the epicardial surface of the myocardium between an occluded and an non-occluded coronary artery, and over a channel in the myocardial wall.

FIG. 3 illustrates an example of cardiac tissue growth following implantation of a sponge-like element on rabbit heart in accordance with an embodiment of the method of the invention. FIG. 3A is a photograph of the excised whole rabbit heart showing tissue growth in and from the implant (boxed area). FIG. 3B is an enlargement of the boxed area of FIG. 3A showing tissue growth and large blood vessel development from the element.

FIG. 4 illustrates an example of cardiac tissue growth following implantation of a sponge-like element on rabbit heart in accordance with an embodiment of the method of the invention. The implant region is bounded by arrows 3, 4, 5 and 6.

FIG. 5 illustrates a rabbit heart following implantation of a sponge-like element without conditioning of the element.

### MODES FOR CARRYING OUT THE INVENTION

In one embodiment, the method of the invention is used to promote or stimulate the growth of blood vessels from the element on the epicardial external surface of cardiac tissue into the cardiac tissue, and in particular, ischaemic cardiac tissue, or in other words, re-vascularisation of ischaemic cardiac tissue.

The ability to promote or stimulate blood vessel growth in and from the element on the epicardial external surface of cardiac tissue permits epicardial development of blood vessels that are able to provide a source of blood flow to intra-myocardial vessels that are impaired in blood flow. The impairment in blood flow may be due to, for example, obstruction of native epicardial vessels.

The method of the invention may be used to improve blood supply to intra-myocardial blood vessels via growth of blood vessels from the element on the epicardial external surface into the tissue to supply the intra-myocardial vessels.

The method of the invention may be used to stimulate or promote collateral circulation from and to large epicardial coronary arteries and/or major epicardial veins. The inventor has also found that by locating an element on the external surface of an occluded artery such that the element extends across the epicardial external surface to contact the surface of an epicardial coronary artery that is non-occluded and wherein the tissue below the non-occluded artery is non-ischaemic and/or non-hypoxic, growth of blood vessels is promoted or stimulated in and from the element which may bridge collateral epicardial vascular development between the non-occluded artery and the occluded artery. This may allow development of new vascular growth in the ischaemic tissue beneath the occluded artery.

The conditioned element may promote or stimulate growth of blood vessels in and from the element into the cardiac tissue. The blood vessels may have well developed adventitia. The blood vessels may comprise smooth muscle cells. The blood vessels may be the result of arteriogenesis.

In one embodiment, the element comprises a sponge-like element. The sponge-like element may be made from polyurethane that is porous, low density, non-degradable, absorptive and resistant to wear and tear by the constant beating of the heart. The sponge-like element has a low inflammatory potential and supports seeding or impregnation with cellular phenotypes. The pores of the sponge-like element may have an average diameter of between 50 and 250 μm in diameter, and are interconnected throughout the element. The sponge-like element may be formed from, for example, foamed polyurethane.

The sponge-like element may be located over the external surface of at least a portion of the epicardial external surface of the heart. The external surface of the heart may be any external surface portion that lies between the AV groove and the apex of the heart. By introducing blood into the sponge-like element to condition the element, tissue growth, in particular angiogenesis and/or arteriogenesis, is promoted or stimulated in and from the element. The inventor has found that when the element is positioned in contact with the epicardial external surface of the heart, blood vessel growth extends from the element and into the tissue when the element is conditioned by introducing blood into the element. The blood vessel growth from the element may be the result of angiogenesis. The blood vessel growth from the element may be the result of arteriogenesis. The blood vessel growth from the element may be the result of angiogenesis and arteriogenesis.

The element may be attached to the external surface of the epicardium in any manner that permits growth of blood vessels from the element. Suitable methods for attaching the element include suturing, tissue glue, or locating the element between the epicardium and the pericardial sac whereby the pericardium holds the element in position.

In one embodiment, the sponge-like element is located on the external surface of the epicardium and blood is introduced into the sponge-like element from the ventricular cavity by way of transmyocardial channels under the element that extend through the ventricular wall from the external surface of the epicardium to the ventricular cavity. The channels in the myocardium permit blood from the ventricular cavity to be introduced into the sponge element on the external surface of the epicardium. Thus, the channel supplies blood to the sponge like element to promote or stimulate growth of blood vessels in and from the element into the tissue to thereby vascularise the tissue. The growth of blood vessels in and from the element may be the result of angiogenesis. The growth of blood vessels in and from the element may be the result of arteriogenesis. The growth of blood vessels in and from the element may be the result of angiogenesis and arteriogenesis.

It will be appreciated by those skilled in the art that the size of the element will depend on the area of tissue to be vascularised. It will also be appreciated that blood need only be introduced to only a portion of the sponge-like element in order to promote or stimulate angiogenesis in and from the element.

By placing the sponge-like element over one or more transmyocardial channel(s), the sponge element seals the channels in an open position and thereby permits continued blood flow to the element from the channel.

The sponge-like element may be located between the epicardial external surface and the pericardial sac. Locating the sponge-like element between the epicardial external surface and the pericardial sac maintains consistent levels of local agents such as growth factors as the pericardium acts as a reservoir for growth factor exchange from the myocardium and vessels in the pericardium.

The pericardium may be open or closed. Adherence of the sponge-like element to the lungs or other tissue may be reduced if the sponge-like element is:

(a) enclosed within the pericardium;

(b) one surface layer of the sponge-like element was closed with no intercommunicating pores,

(c) an artificial pericardial layer over the surface, or material currently used in cardiac surgery to prevent adhesions.

Promoting or stimulating angiogenesis and/or arteriogenesis from the sponge-like element on the external surface of the epicardium results in development of large blood vessel growth into the myocardium. These large blood vessels may link with intramyocardial blood vessels to further improve blood supply to the cardiac tissue. Growth of intramyocardial blood vessels may be promoted or stimulated by forming one or more transmyocardial channels. The growth of intramyocardial blood vessels may be further promoted or stimulated by placement of one or more devices for stimulating or promoting angiogenesis within the transmyocardial channel(s). Examples of such devices are described in, for example, US Patent Application No. 20010004690, US Publication No. US 2002/0032476. U.S. Pat. No. 6,458,092, US Patent Publication No. US 2001/0008969. Transmyocardial channels in the myocardium as mentioned above may also serve to link the two populations of blood vessels.

One or one or more devices for stimulating or promoting angiogenesis as mentioned above may be inserted within the channels to increase the supply of intramyocardial blood vessels to further improve blood supply in the cardiac tissue. The placement of these devices within the channel may result in development of smaller intramyocardial blood vessels. The device within the channel may contact the sponge like element on the epicardial external surface. Alternatively, the device within the channels may be spaced apart from the sponge-like element on the epicardial external surface, whereby angiogenesis within the channel between the sponge-like element and the device may be promoted or stimulated.

In one embodiment, the sponge-like element may be a elongate strip or a sheet located on the external surface of the epicardium over coronary blood vessels that have been obstructed, and in contact with coronary vessels that have not been obstructed, whereby growth of tissue in and from the element may provide blood supply from the unobstructed vessels to intromaycardial blood vessels which were supplied by the obstructed coronary vessel prior to the obstruction. Thus, the sponge-like element may act as a bridge to promote blood vessel growth from an unobstructed coronary artery to regions where a coronary artery occlusion has prevented sufficient blood supply to myocardial tissue.

The sponge-like element may have protrusions extending from at least one surface of the element which may be inserted in the channel created in the myocardium. Blood from the channels may be introduced into the element via the protrusions. The protrusions may serve to anchor the element to the epicardial surface. The protrusions may be in fluid communication with the at least one portion of the element and may serve to receive blood from the channels, and in turn introduce the blood to the at least one portion of the element on the epicardial external surface. The protrusions are unitary with the element, or in other words, the protrusions are an integral part of the element.

Treatment of Infarcted Tissue

The sponge-like element may be located over and in contact with the external surface of an infarct scar to prevent or slow thinning of the scar and/or to increase scar thickness. The scar thickness may be further thickening with additional scar tissue or via another cellular matrix source. It is envisaged that the sponge like element may extend beyond the immediate external surface of the infarct scar to the external surface of non-infarcted tissue. In other words, the element may be located in contact with the epicardial external surface of the infarcted tissue and patent epicardial coronary arteries to thereby increase epicardial blood supply to affected scar regions and the neighboring infarct border zone where the tissue may still be vulnerable to ischeamia.

Typically, channels extending from the epicardial external surface to the ventricular cavity are formed in the infarct scar tissue to perfuse the sponge-like element with blood from the ventricular cavity. The channels created in the infarcted tissue also allow for epicardial blood supply to communicate with an intra-myocardial microcirculation, thereby providing an enhanced microcirculation to the infarct border zone. Devices for stimulating or promoting intramyocardial blood vessels (such as those described in, for example, US Patent. Application No. 20010004690, US Publication No. US 2002/0032476. U.S. Pat. No. 6,458,092, US Patent Publication No. US 2001/0008969) may be placed in the channels to permit blood vessel development between the sponge-like element on the epicardial external surface and the device(s) in the channel.

Growth of tissue in and from the sponge-like element in contact with the epicardial external surface of the infarct scar may be influenced via release of drugs or cell seeding or combination thereof to increase the growth of tissue that promotes a more compliant scar, such as myofibroblasts, smooth muscle cells, skeletal muscle cells, or progenitor cells (i.e. stem cells) that will differentiate into cardiac myocytes. These cells may be placed within the sponge-like element. Cells may be seeded onto the matrix at the time of implantation via cell culture techniques. Alternatively, cells may be harvested at the time of locating the sponge-like element on the epicardial surface and delivered directly into the sponge-like element and/or underlying myocardium or scar tissue at time of locating the element or following location of the element.

Formation of a Wound

The wound may be formed using any methods known in the art. In one embodiment, the wound is formed using radio frequency ablation. Methods of cutting tissue using radiofrequency ablation are described in, for example, Dorwarth, U., et al. (2003) Radiofrequency catheter ablation: different cooled and noncooled electrode systems induce specific lesion geometries and adverse effects profiles, Pacing Clin. Electrophysiol 26(7 Pt 1): 1438-45.

In another embodiment, the wound is formed by cutting with trans-myocardial laser revascularization.

The wound may be formed by parting two adjacent points of the myocardium abutting each other whereby the tissue is teased apart and in so doing creating a channel in the myocardium. Release of the teased tissue would result in closing of the channel, whereby the tissue would spring back to oppose each other and seal the channel opening. This channel would be maintained open in part if the sponge material was placed between the two open sides of the myocardium to thereby seal the sponge between the two opposing sides of the myocardium.

The wound may be formed using a needle or scalpel.

Aortic Wrap or Banding

External aortic wall reinforcement with Dacron graft material for wrapping or banding as a primary method of surgical treatment of aortic aneurysm is associated with aortic wall rupture. (see Dhillon J S, Randhawa G K, Straehley C J, McNamara J J. Late rupture after dacron wrapping of aortic aneurysms, Circulation 1986 September;74(3 Pt 2):I11-4.

The physiological response to banding or wrapping the aorta is to off-load arterial wall tension. Wrapping or banding with non-compliant material in turn induces rapid and extensive atrophy of the arterial media, henceforth causing cross-sectional thinning of the aortic wall (see Ilana M. Bayer; S. Lee Adamson; B. Lowell Langille. Atrophic Remodeling of the Artery-Cuffed Artery. Arteriosclerosis, Thrombosis, and Vascular Biology. 1999;19:1499-1505). Surgeons have targeted the aorta to enhance cardiac output by cyclic compression of the aorta in timing with the cardiac cycle—for example, a peri-aortic counterpulsation jacket that comprises a means including a fluid expansible balloon for compressing a portion of the aorta during diastole (see, for example, PCT/CA90/00390). Likewise aortic counter-compression of the aorta is associated with cross-sectional aortic wall thinning.

Wrapping or banding of at least a portion of the aorta with the sponge-like element on the outside surface of arteries such as the aorta may reduce wall stress and/or affect compliance and/or re-shape a dilated aorta or prevent dilation. The wrapping or banding of the aorta with a sponge-like element may be used to treat aortic aneurysm.

For example, by wrapping at least a portion of the aorta with a sheet of the sponge-like element, the cross-sectional thickness of the aorta may be increased through new vascular wall cross-sectional tissue growth. Blood may be introduced into the element by forming micro-channels through the cross-section of the aortic wall.

Growth Factors and Anti-Coagulation Substances

Different concentrations of growth factors have been purported to influence epicardial vascular growth and intramyocardial angiogenesis differentially. It is purposed that the sponge-like element may be coated with different reagents along the length of the element. For example, the portion of the sponge-like element in close vicinity of an epicardial coronary artery, or that portion of the sponge-like element located in channels, may be coated with growth factors appropriate for further epicardial vascular development. This too may encompass different pore sizes for that part of the sponge located closer to the epicardial surface, typically large pore sizes. Growth factors employed for the lower part of the sponge may be used to promote microcirculation such as capillary growth within the channel.

Substances may be inserted into the wound prior to placement of the sponge-like element. For example, hydrogel containing substances that prevent coagulation or clot formation such as heparin, warfarin and GAS-6 may be inserted in the wound. The hydrogel may permit a sustained release of these substances over time to prevent coagulation within the wound.

Cell Seeding/Tissue Engineering

The sponge-like element allows also for impregnation and growth of seeded or cultured cells such as stem cells in vitro prior to locating the element on the epicardial surface. Alternatively, these cell types could also be delivered to the sponge-like element any time after locating the element by injecting the cells into the sponge-like element using a catheter or syringe delivery system. Typically the cells which are injected into the sponge-like element are suspended in a viscous hydrogel matrix or extracellular matrix such as fibronectin and/or tropelastin (such as, for example, recombinant tropoelastin) and/or vitronectin and/or combinations thereof.

Cells that are either delivered onto the sponge-like element after locating the element or seeded onto the sponge-like element before locating the element will determine its cellular characteristics with regard to tissue growth in and from the element over time.

Cell types could be seeded onto the sponge-like element that provide for the formation of capillaries and blood vessels within the sponge-like element and from the element into the myocardial tissue in the heart. However, such cells would only provide a supplement to cells which grow in and from the element from the tissue, but would not be necessary for growth of tissue in and from the element. Thus, in the absence of impregnation of the sponge-like element with cells derived from sources other than the tissue, the element would support cellular growth from the tissue in contact with the sponge-like element and the tissue from this cellular growth would also have a significant angiogenesis component. This angiogenesis component extends beyond the sponge-like element into the myocardial tissue.

Sponge-like elements that are not seeded, cultured or injected with cells are likely to have varying ratios of cell phenotypes and proteins occupying the complete sponge-like element over time. It is anticipated that these cell and protein populations would consist of myofibroblasts, fibroblasts, smooth muscle cells, pericytes, endothelial cells, collagen subtypes, basement membrane and other cell and/or protein types.

Before locating the sponge-like element onto the epicardial external surface, the sponge-like element may be placed for a few hours in cell culture to promote seeding of the sponge-like element. Specifically, the cell types in culture may be cardiac myocytes or stem cells or progenitor cells that are “spore-like”, or a combination of these cell types. It is envisaged that the stem cells or “spore-like” progenitor cells would differentiate into cardiomyocytes within the matrix of the sponge-like element after locating the element.

Alternatively the sponge-like element could be placed in culture for prolonged periods of time to allow cell attachment and further development of cardiomyocytes within the sponge-like element before locating the element.

Myocytes seeded or cultured onto the sponge-like element would be supported by blood and oxygen diffusion through the sponge-like element following locating of the element onto the epicardial surface.

Myocyte regeneration within the sponge-like element would have an application for engineering new myocardial tissue to areas of the heart that have developed significant fibrosis or scar tissue. Specific regions of interest would be those areas where scar or fibrotic tissue is within sub-epicardial and or transmural regions of the heart. The application of the sponge-like element would promote angiogenesis and or arteriogenesis and support new tissue growth and replacement of scar tissue. Stem cells or stem cell factors may migrate from the supporting sponge-like element to replace scar or fibrosis tissue or promote stem cell migration and subsequent replacement of scar or fibrosis tissue. The sponge-like element could be seeded or cultured using cell types described above and located in areas where epicardial/sub-epicardial fibrosis or scar tissue exists. New tissue can be created to expand the cross-sectional myocardial wall and or replace sub-epicardial fibrosis or scar tissue with cardiac cells and/or cardiomyocytes that are supported by stimulated angiogenesis and or arteriogenesis.

Cardiomyocyte development within the sponge-like element would allow cardiomyocytes to form gap junctions between adjacent cardiomyocytes through connexins, typically connexin-43. Gap junction formation between cardiomyocytes within the sponge-like element and the formation of gap junctions with cardiomyocyte populations in tissue below and in contact with the sponge-like element would promote cardiac electrical stability.

Alternatives to cardiac myocyte regeneration within the sponge-like element would be to deliver or culture other muscle cellular phenotypes within the sponge-like element such as skeletal or smooth cells. Smooth muscle cells express connexin 43 and may form gap junctions with cardiac myocytes at the myocardium/sponge-like element interface. Smooth muscle cells or skeletal muscle cells may be seeded or cultured onto the implanted sponge-like element as described above for cardiomyocytes, stem cells or progenitor cells that are “spore-like”.

Formation of gap junctions between cells within the implanted sponge-like element and at the border between the sponge-like element and tissue promotes homogeneous electrical conduction throughout the conditioned sponge-like element and surrounding myocardial tissue during the cardiac cycle. Advantageously, electrical conductance across the sponge-like element would be expected to be uniform and not to create areas of inhomogeneous conduction. Thus, it is envisaged that the implanted sponge-like element would be unlikely to cause areas of ventricular arrhythmia foci.

As discussed above, the sponge-like element may be used in the method of the invention to support homogeneous electrical conduction and/or cardiac electrical stability. It is envisaged that the method of the invention may be used as an alternative in some circumstances, to currently used ventricular ablation techniques and/or drug treatments for ventricular arrhythmias. The method of the invention may be used as a method to promote cardiac electrical stability. For example, by supporting the replacement of scar or fibrotic tissue, and/or decreasing the risk of scar aneurysm formation, or repairing ventricular wall or scar aneurysm formation that is associated with decreased cardiac electrical stability. In addition, areas of abnormal conduction may be due to ischaemia—therefore restoration of blood supply by promoting angiogenesis by placing the sponge-like element in contact with heart tissue may promote normal cardiac myocyte function/cellular function and a return of homogenous conduction.

Following trans-myocardial laser revascularization (TMLR), location of a portion of the sponge-like element in the resulting channel may assist in the prevention of closing of myocardial channels. The problem of channels closing after transmyocardial revascularization has been noted in the scientificmedical literature previously. TMLR channels typically become scar tissue.

In one embodiment, the inherent absorptive properties of the sponge-like element permit the uptake of growth factors, serum etc and its release over time to surrounding tissues when implanted.

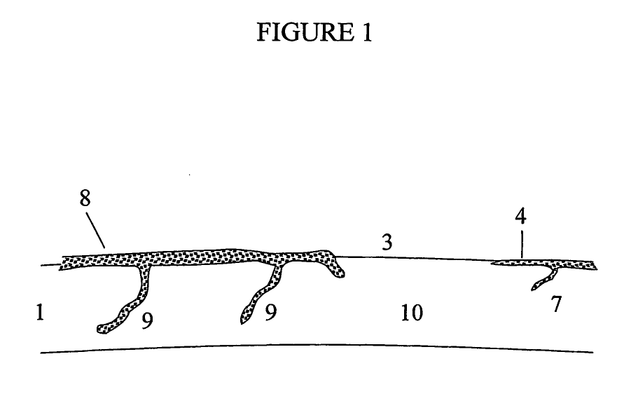

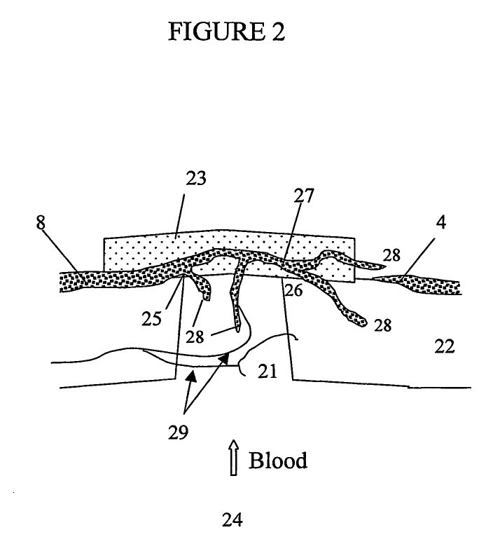

FIG. 1 illustrates a cross-section of cardiac tissue without applying the method of the invention, and FIG. 2 illustrates embodiment of the method of the invention applied to cardiac tissue.

Referring to FIG. 1, there is depicted a schematic representation of a cross-section of the myocardial ventricular wall (1) with normal un-occluded coronary artery (8) on the epicardial surface (3) having epicardial collateral branches (9) extending into the mid-myocardium (10). There is also depicted a coronary artery on the epicardial surface that has become occluded (4), narrowed and from which little collateral circulation is present to supply blood to the sub-epicardial muscle below. The tissue within the area under the occluded artery (7) would be rendered ischaemic.

FIG. 2 is a schematic representation of the same portion of myocardium as FIG. 1 in which a channel (21) has been formed in the myocardium (22) and a sponge-like element (23) has been located on the external surface of the epicardium in accordance with an embodiment of the present invention. Blood from the ventricular cavity (24) is introduced into the sponge-like element. The element seals the channel in an open configuration at positions (25) and (26). Introduction of blood into the element conditions the element and results in the growth of blood vessels in (27) and from (28) the element. The sponge-like element also overlays and extends from the un-occluded coronary artery (8) to the occluded artery (4). The conditioned sponge-like element stimulates or promotes blood vessel growth in and from the element, through the myocardial channel and through the muscle tissue underlying the occluded artery to vascularise the ischaemic tissue and create a network of blood vessels (28, 29).

### EXAMPLE

A. Surgical Attachment of the Sponge-Like Element to the Heart

A sponge-like element (polyurethane polycarbonate biomaterial scaffold) was sutured onto the surface of the left ventricle in 5 Adult New Zealand White Rabbits.

Three groups of rabbits were formed:

Group I-2 rabbits in which the sponge-like element was implanted by suturing directly onto the external surface of the exposed ventricular epicardium but in which no communicating channels were formed.

Group II—2 rabbits in which the sponge-like element was implanted by suturing onto the external surface of the epicardium, but prior to suturing the sponge-like element onto the external surface of the epicardium, a communicating blood channel was made between the epicardial external surface and the ventricular cavity, thus allowing for blood to spurt from the channel (wound). Following the creation of the channel (wound) the sponge-like element was directly placed over the wound to allow blood to interact with the sponge-like element. The sponge-like element was secured to the external surface of the epicardium by placement of a suture. In doing so the sponge-like element occluded the escape of blood from the channel into the chest cavity and the sponge-like element itself was in direct communication with a blood source.

Group III—one rabbit in which a channel was made at a site remote from the implantation of the sponge-like element. This served as a control for the effect of the blood channel itself.

To carry out sponge-like element implantations surgically, rabbits were anesthetised with intravenous thiopentone (1.25%) 20 mg/kg. Rabbits were intubated with a 3-0 uncuffed endotracheal tube and hand ventilated via a bag connected to a bain circuit. Hand ventilation was only required when the chest was opened. Anesthesia was maintained with halothane 2% and supplemented with oxygen 1L-2L/min. All animals received intravenous cefazoline (100 mg) preoperatively. A thoracotomy was performed in the left 4^{th} intercostal space under sterile conditions. The pericardium was incised and the left circumflex coronary artery could be visualized. The sponge-like element was sutured directly over an external surface area of epicardium in close proximity to the coronary artery between apex and base. When suturing the sponge-like element to the external surface of the heart, care was taken not to place a suture through any coronary artery that had been visualized.

Creation of epicardial trans-myocardial channels was made using an 18-gauge needle. The 18-gauge needle was vertically positioned over epicardial external surface of the free wall of the left ventricle and advanced into and through the myocardial wall until blood started to ooze out of the lumen of the needle. This indicated that the needle had advanced into the ventricular cavity. When the needle was removed, a hole with blood spurting from it was identified. If such a channel is not observed the procedure was repeated. Care was taken not to create a channel through a coronary vessel.

When the trans-myocardial channel was created, the sponge-like element was sutured to the external surface of the epicardium such that the sponge-like element was sutured over the external surface of the hole and occluded the channel. The position of the sponge-like element implant permits communication with a blood supply and was verified by a red color of the implant. The implant was soaking up blood perfusing it. The sponge-like element was of greater size than the created 18-guage channel and thus the sponge-like element was also in contact with the external surface of the epicardium.

Following sponge-like element implantation, a chest tube was inserted and the wound closed in layers. Animals were recovered and kept alive for 8-10 weeks. Following 8-10 weeks of sponge-like element implantation the rabbits were euthanized. The chest opened, the heart removed and washed in ice-cold saline. The heart was then emersion fixed in Zamboni's [Stefanini, M., De Martino, C., and Zamboni, L. (1967) Fixation of ejaculated spermatozoa for electron microscopy. Nature 216: 173-174.] for 6 hours and then placed in 70% alcohol.

B. Macroscopic Observations of Animals with Attached Sponge-like Element.

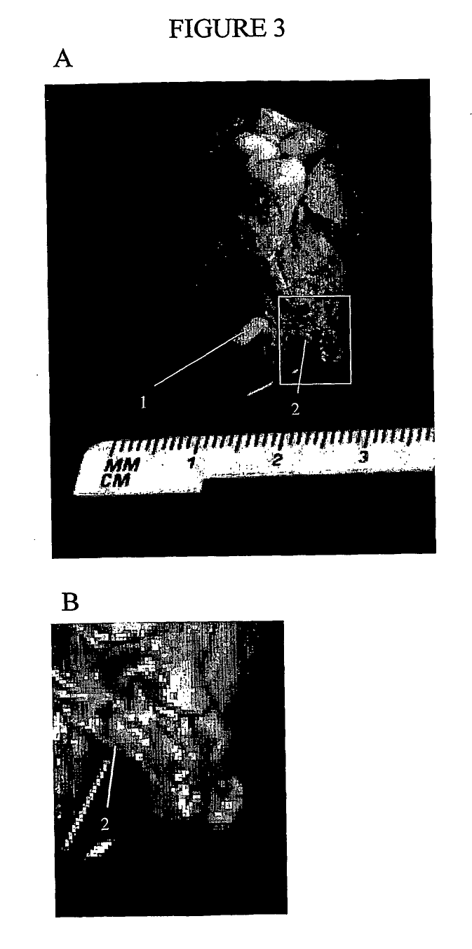

All the New Zealand White rabbits from Group II above in which an epicardial sponge-like element implant (for 8-10 weeks) communicates with blood channels were found to promote superior tissue outgrowth, i.e. beyond the dimensions of the implanted biomaterial matrix. In one of the 2 implants, significant vascular development could be visualized on the surface of the new tissue formed (see FIGS. 3 A and B).

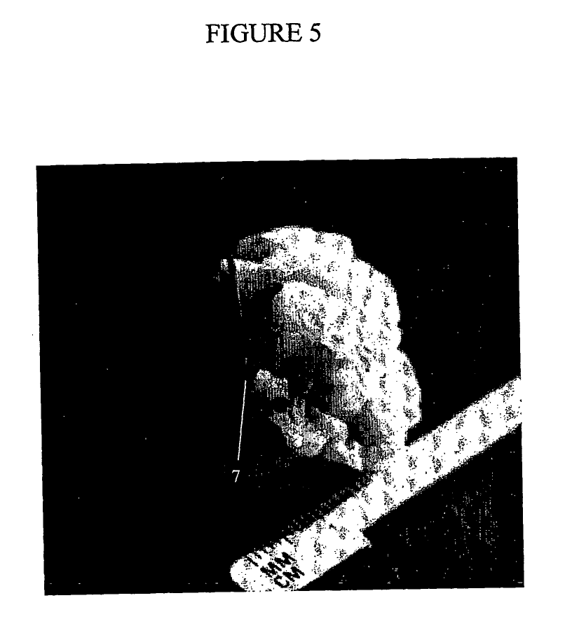

In all the rabbits from Group I in which channels were not created and implants were simply sutured onto the surface of the myocardium (for 8-10 weeks), only limited tissue outgrowth was observed and the amount of new tissue was not greater than the implant (FIG. 5). Rather there was some tissue in-growth within part of the sponge-like element. The tissue growth did not extend significantly beyond the implant.

In the one rabbit from Group III in which a channel was formed remote from the implant, no tissue growth was observed at the site of the channel on the surface of the epicardium.

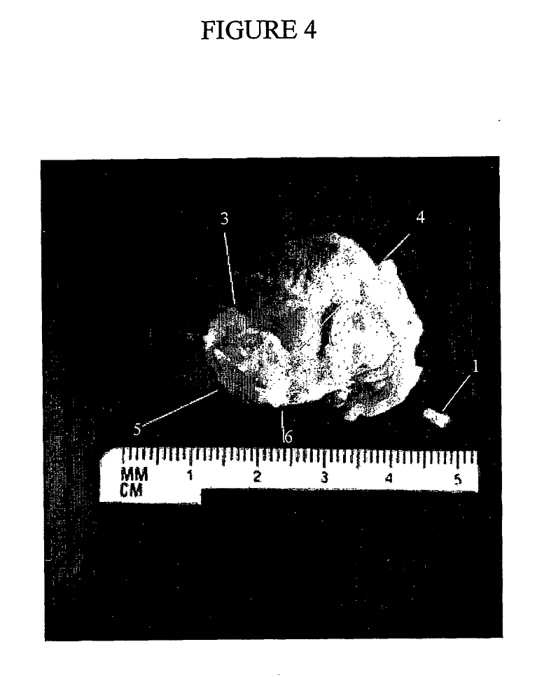

FIGS. 3 and 4 represent chronic implants in which the sponge-like element was implanted in communication with a vascular channel.

FIG. 5 represents a chronic sponge-like element implantation where the implant was not in communication with a vascular channel. Note that in FIG. 5 the sponge-like element had been implanted near the marker needle (at the position marked 7) to show its position. Aspects of the sponge-like element implant in FIG. 5 still resemble a non-implanted sponge-like element.

FIG. 3 depicts both the sponge-like element implant in situ and the significant new tissue growth surrounding the sponge-like element implant. For comparison, a sponge-like element of a similar size to the original implant has been placed on the surface of the myocardium (1). A similar case is depicted in FIG. 4 where the non-implanted sponge-like element for comparative purposes (1) is next to the ruler (scale guide), and significant tissue growth is depicted on the surface of the heart—3, 4, 5 and 6 on the photograph demonstrate the boundaries for new tissue growth following a sponge-like element implant.

The new tissue growth and implant area for FIG. 3A have been enlarged in FIG. 3B and the arrow (2) depicts a large blood vessel penetrating the tissue surrounding the biomaterial implant and surrounding epicardium. This case (FIG. 3 A and B) demonstrates new macroscopic or large blood vessels.

C. Histological Observations

Further histological examination of the sponge-like element implants was undertaken in cases where vascular blood vessel development was observed on macroscopic inspection of the heart (FIGS. 3A and B). A cross-section H&E light microscope tissue section was examined. This cross section included the sponge-like element implant, new tissue growth and the complete left-ventricular cross-sectional wall. The histological section showed tissue ingrowth into and beyond the sponge-like element. Significant angiogenesis within the sponge-like element was observed with the formation of numerous capillaries containing red blood cells. Some inflammatory markers were found within the sponge-like element matrix of the implant. In the sub-epicardium just beneath the sponge-like element implant there seemed to a high proportion of large blood vessels the size of coronary arteries and their collaterals. This density of vascular development within the sub-epicardium was greater than any other scanned region of the cross-sectional ventricular wall that did not have an overlying biomaterial implant, additionally the density seemed greater than in a control heart.

It is to be understood that a reference herein to a prior art publication does not constitute an admission that the publication forms a part of the common general knowledge in the art in Australia, or any other country.

1. A method for promoting or stimulating growth of tissue comprising the following steps:
providing an element which is adapted to receive blood;
locating the element in contact with the tissue with at least one portion of the element extending away from an external surface of the tissue; and
conditioning the element by introducing blood into said at least one portion whereby, when located in contact with the tissue, the conditioned element is arranged to stimulate or promote growth of tissue in and from the at least one portion.

2. The method of claim 1 wherein angiogenesis is stimulated or promoted in the at least one portion.

3-38. (canceled)

39. The method of claim 1 wherein angiogenesis is stimulated or promoted from the at least one portion.

40. The method of claim 1 wherein arteriogenesis is stimulated or promoted in the at least one portion.

41. The method of claim 1 wherein arteriogenesis is stimulated of promoted from the at least one portion.

42. The method of claim 1 wherein at least one surface of the at least one portion of the element is in contact with the external surface of the tissue.

43. The method of claim 1 wherein the blood is introduced into said at least one portion from a wound.

44. The method of claim 43 wherein the wound is formed in the tissue.

45. The method of claim 44 wherein the element introduces blood into the at least one portion by absorption, capillary action or suction.

46. The method of claim 1 wherein the element is a sponge-like structure.

47. The method of claim 46 wherein the sponge-like structure is formed from polyurethane.

48. The method of claim 1 wherein the at least one portion of the element is a sheet-like structure and at least one surface of the at least one portion is in contact with a portion of the external surface of the tissue.

49. The method of claim 1 wherein the element is an elongate member having at least one surface of the at least one portion in contact with a portion of the external surface of the tissue.

50. The method of claim 1 wherein the at least one portion is in contact with at least one wound in the tissue.

51. The method of claim 1 wherein at least one surface of the at least one portion extends along and in contact with the external surface of the tissue between and in contact with at least two wounds.

52. The method of claim 1 wherein at least one surface of the at least one portion is in contact with a portion of the external surface of the tissue, and at least one other portion is in contact with one or more wounds to receive blood from the wounds, the at least one other portion being in fluid communication with the at least one portion.

53. The method of claim 1 wherein the tissue is muscle tissue.

54. The method of claim 53 wherein the tissue is cardiac tissue.

55. The method of claim 54 wherein the cardiac tissue is ischaemic cardiac tissue.

56. The method of claim 1 wherein the tissue is pancreatic tissue.

57. The method of claim 1 wherein the tissue is an aorta.

58. A method for treating ischaemic tissue comprising the following steps:
providing an element which is adapted to receive blood;
locating the element in contact with the ischaemic tissue with at least one portion of the element extending away from an external surface of the tissue; and
conditioning the element by introducing blood into said at least one portion whereby, when located in contact with the ischaemic tissue, the conditioned element is arranged to stimulate or promote growth of tissue in and from the at least one portion.

59. A method for treating ischaemic cardiac tissue comprising the following steps:
providing an element which is adapted to receive blood;
locating the element with at least one portion of the element in contact with the epicardial external surface of the ischaemic cardiac tissue; and
conditioning the element by introducing blood into the at least one portion wherein the growth of tissue is stimulated or promoted in and from the at least one portion into the ischeamic cardiac tissue.

60. A method for treating ischaemic heart disease comprising the following steps:
providing an element which is adapted to receive blood;
locating the element with at least one portion of the element in contact with the epicardial external surface of ischaemic cardiac tissue; and
conditioning the element by introducing blood into the at least one portion wherein the growth of tissue is stimulated or promoted in and from the at least one portion into the ischaemic cardiac tissue.

61. A method for treating myocardial infarction comprising the following steps:
providing an element which is adapted to receive blood;
locating the element with at least one portion of the element in contact with an epicardial external surface of infarcted myocardial tissue; and
conditioning the element by introducing blood into said at least one portion to stimulate or promote growth of tissue in and from the at least one portion into the infracted myocardial tissue.

62. A method for promoting or stimulating growth of tissue comprising the following steps:
providing an element which is adapted to receive blood;
locating the element in contact with the tissue with at least one portion of the element extending away from the surface of the tissue; and
conditioning the element by introducing blood into said at least one portion whereby, when located in contact with the tissue, the conditioned element is arranged to stimulate or promote angiogenesis in and from the at least one portion.

63. A method for promoting or stimulating growth of tissue comprising the following steps:
providing an element which is adapted to receive blood;
locating the element in contact with the tissue with at least one portion of the element extending away from the external surface of the tissue; and
conditioning the element by introducing blood into said at least one portion whereby, when located in contact with the tissue, the conditioned element is arranged to stimulate or promote arteriogenesis in and from the at least one portion.

64. A method for promoting or stimulating growth of tissue comprising the following steps:
providing an element which is adapted to receive blood;
locating the element in contact with the tissue with at least one portion of the element extending away from the external surface of the tissue; and
conditioning the element by introducing blood into said at least one portion whereby, when located in contact with the tissue, the conditioned element is arranged to stimulate or promote angiogenesis and arteriogenesis in and from the at least one portion.

65. A method for promoting or stimulating growth of tissue comprising the following steps:
providing an element which is adapted to receive blood;
locating the element in contact with the tissue with at least one portion of the element extending away from the external surface of the tissue; and
conditioning the element by introducing blood into said at least one portion whereby, when located in contact with the tissue, the conditioned element is arranged to stimulate or promote angiogenesis in, and arteriogenesis from, the at least one portion.

66. A method for promoting or stimulating growth of tissue comprising the following steps:
providing an element which is adapted to receive blood;
locating the element in contact with the tissue with at least one portion of the element extending away from the external surface of the tissue; and
conditioning the element by introducing blood into said at least one portion whereby, when located in contact with the tissue, the conditioned element is arranged to stimulate or promote arteriogenesis in, and angiogenesis from, the at least one portion.

67. A growth promoting or growth stimulating element comprising a mesh formed from knitted or woven yarn or fibre, wherein the mesh is coated with polyurethane or polyurethane polycarbonate.

68. The element of claim 67 wherein the yarn or fibre is formed from one or more materials selected from the group consisting of cotton, linen, silk, knitted silkworm silk, insect silk, a polyamide, polyhexamethylene sebacamide, polycapramide, polydodecanamide, polyhexamethylene isophthalamide, a polyester, a fluoropolymer, a polyolefin, and polyethylene.

69. The element of claim 67 wherein the yarn or fibre is formed from one or more materials selected from the group consisting of an aliphatic polyester, a glycolide, glycolic acid, epsilon-caprolactone, p-dioxanone (1,4-dioxan-2-one), trimethylene carbonate (1,3-dioxan-2-one), an alkyl derivative of trimethylene carbonate, delta-valerolactone, beta-butyrolactone, gamma-butyrolactone, epsilon-decalactone, hydroxybutyrate, hydroxyvalerate, 1,4-dioxepan-2-one, 1,5,8,12-tetraoxacyclotetradecane-7,14-dione, 1,5-dioxepan-2-one, 6,6-dimethyl-1,4-dioxan-2-one, polyglactin 910 (Vicryl), polyglycolic acid (Dexon), and a blend of any of the foregoing polymers.

70. The element of claim 67 wherein the mesh is coated with the polyurethane or polyurethane polycarbonate after the mesh is woven or knitted.

71. The method of claim 1 wherein the element comprises a mesh formed from knitted or woven yarn, wherein the mesh or yarn is coated with polyuyrethane or polyurethane polycarbonate.

72. A growth promoting or stimulating element adapted to receive blood, the element comprising a porous matrix comprising an polymer or polymer coating, the matrix being adapted to receive blood and sized to fit within a wound in a tissue, such that when the element is located in contact with the tissue with at least one portion of the element extending away from an external surface of the tissue, and when the element is conditioned by introducing blood into at least one portion of the element, growth of the tissue is promoted or stimulated in and from the at least one portion of the element.
